SEQUENCE LISTING (22 sequences)

SEQ ID NO: 1 - DNA, 38 bp, synthetic construct
  source 1..38: mol_type = other DNA
tgtaaaacgacggccagtcttcccacaggcgcctatag

SEQ ID NO: 2 - DNA, 20 bp, synthetic construct
  source 1..20: mol_type = other DNA
aactgagacggagcaatgca

SEQ ID NO: 3 - DNA, 276 bp, synthetic construct
  source 1..276: mol_type = other DNA
cttcccacaggcgcctatagttgaagggctgagaggcagctgtggagatgaagaaaattacataccatcaccaacaatgacgagcttccttcctgatctattgaattaagagagaaaaagaagagaaagaaagagagaattgacatacagggaattctgagaggccatttgagagagaagatgagggagagagagagagaaagaaagagttgagctatggaagcagaccaagcagaacgtcacagccttcggtcgctgcattgctccgtctcagtt

SEQ ID NO: 4 - DNA, 278 bp, synthetic construct
  source 1..278: mol_type = other DNA
cttcccacaggcgcctatagttgaagggctgagaggcagctgtggagatgaagaaaattacataccatcaccaacaatgacgagcttccttcctgatctattgaattaagagagaaaaagaagagaaagaaagagagaattgacatacagggaattctgagaggccatttgagagagaagatgagggagagagagagagagaaagaaagagttgagctatggaagcagaccaagcagaacgtcacagccttcggtcgctgcattgctccgtctcagtt

SEQ ID NO: 5 - DNA, 282 bp, synthetic construct
  source 1..282: mol_type = other DNA
cttcccacaggcgcctatagttgaagggctgagaggcagctgtggagatgaagaaaattacataccatcaccaacaatgacgagcttccttcctgatctattgaattaagagagaaaaagaagagaaagaaagagagaattgacatacagggaattctgagaggccatttgagagagaagatgagggagagagagagagagagagaaagaaagagttgagctatggaagcagaccaagcagaacgtcacagccttcggtcgctgcattgctccgtctcagtt

SEQ ID NO: 6 - DNA, 38 bp, synthetic construct
  source 1..38: mol_type = other DNA
tgtaaaacgacggccagtggcacgtggttgaggatgta

SEQ ID NO: 7 - DNA, 20 bp, synthetic construct
  source 1..20: mol_type = other DNA
ttcctattactcgcgggcac

SEQ ID NO: 8 - DNA, 225 bp, synthetic construct
  source 1..225: mol_type = other DNA
ggcacgtggttgaggatgtatttgatgtcgacaagtattcggacttgtgtgtgtgtgtgtgtgagagtgatttagaacagggatacttaagatacatgaaactgatgtggagacgacaagagttgacgatgaatggataaatgaatgatgggacgaggatgcacaatccgcgaatgtggattgcgccatgtgacgcaacgagggcgtgcccgcgagtaataggaa

SEQ ID NO: 9 - DNA, 229 bp, synthetic construct
  source 1..229: mol_type = other DNA
ggcacgtggttgaggatgtatttgatgtcgacaagtattcggacttgtgtgtgtgtgtgtgtgtgtgagagtgatttagaacagggatacttaagatacatgaaactgatgtggagacgacaagagttgacgatgaatggataaatgaatgatgggacgaggatgcacaatccgcgaatgtggattgcgccatgtgacgcaacgagggcgtgcccgcgagtaataggaa

SEQ ID NO: 10 - DNA, 231 bp, synthetic construct
  source 1..231: mol_type = other DNA
ggcacgtggttgaggatgtatttgatgtcgacaagtattcggacttgtgtgtgtgtgtgtgtgtgtgtgagagtgatttagaacagggatacttaagatacatgaaactgatgtggagacgacaagagttgacgatgaatggataaatgaatgatgggacgaggatgcacaatccgcgaatgtggattgcgccatgtgacgcaacgagggcgtgcccgcgagtaataggaa

SEQ ID NO: 11 - DNA, 38 bp, synthetic construct
  source 1..38: mol_type = other DNA
tgtaaaacgacggccagttgggtgatgaattggagggc

SEQ ID NO: 12 - DNA, 20 bp, synthetic construct
  source 1..20: mol_type = other DNA
ccaacaacgcgtcacaactc

SEQ ID NO: 13 - DNA, 273 bp, synthetic construct
  source 1..273: mol_type = other DNA
tgggtgatgaattggagggctcgtatgtgggcgtttattaagatgtcatctgatgaggtagggtcgagacaccattcttcgaaagaagggagaccgggtgcatcccatatttgggaaagtgagtatctagctaggtcttttgcagccattgatgttgaacttgagcctgcaggagatgatgatgatgatgatgatgatggtgtggttattatacagtgtgggcattgacaatggaaacggtattggtcgtatagagttgtgacgcgttgttgg

SEQ ID NO: 14 - DNA, 276 bp, synthetic construct
  source 1..276: mol_type = other DNA
tgggtgatgaattggagggctcgtatgtgggcgtttattaagatgtcatctgatgaggtagggtcgagacaccattcttcgaaagaagggagaccgggtgcatcccatatttgggaaagtgagtatctagctaggtcttttgcagccattgatgttgaacttgagcctgcaggagatgatgatgatgatgatgatgatgatggtgtggttattatacagtgtgggcattgacaatggaaacggtattggtcgtatagagttgtgacgcgttgttgg

SEQ ID NO: 15 - DNA, 279 bp, synthetic construct
  source 1..279: mol_type = other DNA
tgggtgatgaattggagggctcgtatgtgggcgtttattaagatgtcatctgatgaggtagggtcgagacaccattcttcgaaagaagggagaccgggtgcatcccatatttgggaaagtgagtatctagctaggtcttttgcagccattgatgttgaacttgagcctgcaggagatgatgatgatgatgatgatgatgatgatggtgtggttattatacagtgtgggcattgacaatggaaacggtattggtcgtatagagttgtgacgcgttgttgg

SEQ ID NO: 16 - DNA, 282 bp, synthetic construct
  source 1..282: mol_type = other DNA
tgggtgatgaattggagggctcgtatgtgggcgtttattaagatgtcatctgatgaggtagggtcgagacaccattcttcgaaagaagggagaccgggtgcatcccatatttgggaaagtgagtatctagctaggtcttttgcagccattgatgttgaacttgagcctgcaggagatgatgatgatgatgatgatgatgatgatgatggtgtggttattatacagtgtgggcattgacaatggaaacggtattggtcgtatagagttgtgacgcgttgttgg

SEQ ID NO: 17 - DNA, 285 bp, synthetic construct
  source 1..285: mol_type = other DNA
tgggtgatgaattggagggctcgtatgtgggcgtttattaagatgtcatctgatgaggtagggtcgagacaccattcttcgaaagaagggagaccgggtgcatcccatatttgggaaagtgagtatctagctaggtcttttgcagccattgatgttgaacttgagcctgcaggagatgatgatgatgatgatgatgatgatgatgatgatggtgtggttattatacagtgtgggcattgacaatggaaacggtattggtcgtatagagttgtgacgcgttgttgg

SEQ ID NO: 18 - DNA, 38 bp, synthetic construct
  source 1..38: mol_type = other DNA
tgtaaaacgacggccagtagcctgtccgtgttatgtgc

SEQ ID NO: 19 - DNA, 20 bp, synthetic construct
  source 1..20: mol_type = other DNA
ccaggagaagtacacgaggc

SEQ ID NO: 20 - DNA, 260 bp, synthetic construct
  source 1..260: mol_type = other DNA
agcctgtccgtgttatgtgcaaacggaaacatagtaaaaataggtattactgtacatggtattaatttattttgtgaaatgttagtgcaatgatatatatatatataagtactgtagatcatctacttataaaaggtgatatttgacattagcagtacacgtatagcgaataccagtttgctgtcatgagaaagacgagaagatatcaagctactttaatgataccgaatacgttgaaacgcctcgtgtacttctcctgg

SEQ ID NO: 21 - DNA, 262 bp, synthetic construct
  source 1..262: mol_type = other DNA
agcctgtccgtgttatgtgcaaacggaaacatagtaaaaataggtattactgtacatggtattaatttattttgtgaaatgttagtgcaatgatatatatatatatataagtactgtagatcatctacttataaaaggtgatatttgacattagcagtacacgtatagcgaataccagtttgctgtcatgagaaagacgagaagatatcaagctactttaatgataccgaatacgttgaaacgcctcgtgtacttctcctgg

SEQ ID NO: 22 - DNA, 264 bp, synthetic construct
  source 1..264: mol_type = other DNA
agcctgtccgtgttatgtgcaaacggaaacatagtaaaaataggtattactgtacatggtattaatttattttgtgaaatgttagtgcaatgatatatatatatatatataagtactgtagatcatctacttataaaaggtgatatttgacattagcagtacacgtatagcgaataccagtttgctgtcatgagaaagacgagaagatatcaagctactttaatgataccgaatacgttgaaacgcctcgtgtacttctcctgg

(19) United States
(12) Patent Application Publication
(10) Pub. No.: US 2024/0218465 A1
(43) Pub. Date: Jul. 4, 2024

(54) DNA BARCODE FOR RECOGNIZING ORIGINAL PLACE OF FLOCCULARIA LUTEOVIRENS

(71) Applicant: XIZANG VOCATIONAL TECHNICAL COLLEGE, LHASA (CN)

(72) Inventors: MANJUN YANG, LHASA (CN); CAIXIA ZHANG, LHASA (CN); YISHUI CAI, LHASA (CN)

(21) Appl. No.: 18/418,420

(22) Filed: Jan. 22, 2024

Related U.S. Application Data
(63) Continuation of application No. PCT/CN2022/110000, filed on Aug. 3, 2022.

(30) Foreign Application Priority Data
Nov. 19, 2021 (CN) 202111401642.9

(51) Int. Cl. C12Q 1/6895; C12Q 1/6806; C12Q 1/686; G01N 27/447

(52) U.S. Cl. CPC C12Q 1/6895; C12Q 1/6806; C12Q 1/686; G01N 27/44726; C12Q 2600/156

(57) ABSTRACT

The present invention discloses a DNA barcode, a primer group and application for recognizing an original place of Floccularia luteovirens, belonging to the technical field of screening of edible fungi germplasm resources. Compared with a traditional breeding method and other existing DNA barcode technologies, the present invention has the advantages of time saving, labor saving, money saving, accuracy and high efficiency, plays a positive role in original place identification and genetic breeding of high-quality Floccularia luteovirens, and also provides an effective method for identification and protection of germplasm resources.

### SEQUENCE LISTING

The instant application contains a Sequence Listing which has been submitted electronically in ST.26 format and is hereby incorporated by reference in its entirety. Said ST.26 copy, created on Jan. 18, 2024, is named Sequence Listing.xml and is 23,754 bytes in size.

### TECHNICAL FIELD

The present invention relates to the technical field of screening of edible fungi germplasm resources, in particular to a DNA barcode, a primer group and application for recognizing an original place of Floccularia luteovirens.

### BACKGROUND

Floccularia luteovirens in a gold yellow color, also known as yellow mushroom and golden mushroom, is a high-quality edible fungus with a unique flavor, which cannot be cultivated artificially at present. Wild Floccularia luteovirens is mainly distributed in Qinghai-Tibet Plateau; and the main producing areas include Dangxiong County of Tibet Autonomous Region, Qilian County of Qinghai Province and Shiqu County of Sichuan Province, and the quality of these three main producing areas is the best. In different producing places, Floccularia luteovirens has different nutritional values, different flavors, different biological activities and different market prices. In the past, the breeding of Floccularia luteovirens was mainly determined by a morphological method combined with beneficial content indexes. However, due to the influence of the special climate environment of Qinghai-Tibet Plateau, the phenomena of different objects with the same name and the same object with different names often appeared to Floccularia luteovirens produced in different areas, so morphological identification was difficult to realize effective distinguishment. In addition, due to high altitude of main producing areas thereof, it is also difficult to collect samples.

A DNA barcode molecular identification technology is a molecular biology technology based on DNA barcodes (conserved and stable genetic DNA sequences in a genome) to recognize and identify species and excellent quality. It is the effective supplement and expansion of traditional breeding methods, and can accurately and effectively identify samples when samples are incomplete in morphologies or lack morphological structures (processed products such as powder, etc.). In order to realize effective development and utilization of Floccularia luteovirens, it is particularly important and urgent to screen different producing areas of Floccularia luteovirens strains with assistance of the DNA barcode molecular identification technology. In the existing DNA barcode technology, non-coding regions or conserved gene sequences in ITS (internal transcribed spacer in ribosomal RNA) and mitochondria are mainly used for species object identification; the operation of restriction fragment length polymorphism (RFLP) is very complicated; reliability and repeatability of results are poor; random amplified polymorphic DNA (RAPD) is easily disturbed, which requires a high technical level of operators and is difficult to popularize in assisted breeding; and single nucleotide polymorphism (SNP) has high requirements for equipment and high cost.

Therefore, in view of shortcomings of traditional breeding methods that the breeding of Floccularia luteovirens strains is not accurate enough, time-consuming and labor-consuming, an urgent problem to be solved by those skilled in the art is how to provide a DNA barcode that can accurately and quickly identify an original place of Floccularia luteovirens.

### SUMMARY

In view of this, the present invention provides a DNA barcode and a primer group for recognizing an original place of Floccularia luteovirens, which can quickly and accurately identify an original place of Floccularia luteovirens, and provides a favorable auxiliary means for breeding high-quality Floccularia luteovirens.

In order to achieve the above purpose, the present invention adopts the following technical solution:

A DNA barcode for recognizing an original place of Floccularia luteovirens, wherein a nucleotide sequence of the DNA barcode includes one or more of:
SEQ ID NO:4,
and/or SEQ ID NO:5,
and/or a combination of SEQ ID NO:3 and SEQ ID NO:4,
and/or a combination of SEQ ID NO:8 and SEQ ID NO:9,
and/or SEQ ID NO:8,
and/or SEQ ID NO:10,
and/or a combination of SEQ ID NO:13 and SEQ ID NO:15,
and/or a combination of SEQ ID NO:13, SEQ ID NO:15 and SEQ ID NO:17,
and/or a combination of SEQ ID NO:13, SEQ ID NO: 14, SEQ ID NO: 15 and SEQ ID NO:16,
and/or a combination of SEQ ID NO:20 and SEQ ID NO:22,
and/or a combination of SEQ ID NO:20, SEQ ID NO:21 and SEQ ID NO:22,
and/or a combination of SEQ ID NO:20 and SEQ ID NO:21.

According to the present invention, based on all simple sequence repeats (SSR) in the whole genome of Floccularia luteovirens, fluorescent PCR amplification is carried out; a DNA barcode which effectively corresponds to an original place is established; and effective recognition of the original place of Floccularia luteovirens can be realized.

Another purpose of the present invention is to provide a primer group for amplifying the DNA barcode for recognizing the original place of Floccularia luteovirens, wherein a nucleotide sequence of the primer group includes one or more groups of:
SEQ ID NO: 1 and SEQ ID NO:2,
and/or SEQ ID NO:6 and SEQ ID NO:7,
and/or SEQ ID NO: 11 and SEQ ID NO: 12,
and/or SEQ ID NO: 18 and SEQ ID NO: 19.

As a preferred technical solution of the present invention, the nucleotide sequence of the primer group includes:
such as SEQ ID NO: 1 and SEQ ID NO:2,
and SEQ ID NO:6 and SEQ ID NO:7,
and SEQ ID NO: 11 and SEQ ID NO: 12,
and SEQ ID NO: 18 and SEQ ID NO: 19.

Different primer groups of the present invention can be used alone or in combination to recognize the original place of Floccularia luteovirens; and when all primer groups are used together, the screening accuracy is the highest.

Another purpose of the present invention is to provide a method for recognizing an original place of Floccularia luteovirens, which comprises the following steps:
S1, extracting genomic DNA of a sample to be tested;
S2, taking the genomic DNA in S1 as a template, and performing a fluorescent PCR amplification reaction on one or more groups of the above primers to obtain an amplification product;
S3, detecting the amplification product of S2 by capillary fluorescence electrophoresis, and judging by the number of fragments, the number of SSR loci, SSR repetitive elements and repeating times of the amplification product.

As a preferred technical solution of the present invention, a judgment standard in step S3 is:
(1) A DNA barcode of Floccularia luteovirens with Qilian County of Qinghai Province as the original place is characterized in that: the primer group of SEQ ID NO:1 and SEQ ID NO:2 is amplified to obtain a 278 bp fragment containing 8 GA repetitive elements;
and/or the primer group of SEQ ID NO:6 and SEQ ID NO:7 is amplified to obtain a 225 bp fragment containing 9 TG repetitive elements and a 229 bp fragment containing 11 TG repetitive elements;
and/or the primer group of SEQ ID NO:11 and SEQ ID NO: 12 is amplified to obtain a 273 bp fragment containing 8 GAT repetitive elements and a 279 bp fragment containing 10 GAT repetitive elements;
and/or the primer group of SEQ ID NO: 18 and SEQ ID NO: 19 is amplified to obtain a 260 bp fragment containing 7 AT repetitive elements and a 264 bp fragment containing 9 AT repetitive elements.
(2) A DNA barcode of Floccularia luteovirens with Shiqu County of Sichuan Province as the original place is characterized in that: the primer group of SEQ ID NO:1 and SEQ ID NO:2 is amplified to obtain a 282 bp fragment containing 10 GA repetitive elements;
and/or the primer group of SEQ ID NO:6 and SEQ ID NO:7 is amplified to obtain a 225 bp fragment containing 9 TG repetitive elements;
and/or the primer group of SEQ ID NO: 11 and SEQ ID NO: 12 is amplified to obtain a 273 bp fragment containing 8 GAT repetitive elements, a 279 bp fragment containing 10 GAT repetitive elements and a 285 bp fragment containing 12 GAT repetitive elements;
and/or the primer group of SEQ ID NO: 18 and SEQ ID NO:19 is amplified to obtain a 260 bp fragment containing 7 AT repetitive elements, a 262 bp fragment containing 8 AT repetitive elements and a 264 bp fragment containing 9 AT repetitive elements.
(3) A DNA barcode of Floccularia luteovirens with Dangxiong County of Tibet Autonomous Region as the original place is characterized in that: the primer group of SEQ ID NO:1 and SEQ ID NO:2 is amplified to obtain a 276 bp fragment containing 7 GA repetitive elements, and a 278 bp fragment containing 8 GA repetitive elements;
and/or the primer group of SEQ ID NO:6 and SEQ ID NO:7 is amplified to obtain a 231 bp fragment containing 12 TG repetitive elements;
and/or the primer group of SEQ ID NO: 11 and SEQ ID NO: 12 is amplified to obtain a 273 bp fragment containing 8 GAT repetitive elements, a 276 bp fragment containing 9 GAT repetitive elements, a 279 bp fragment containing 10 GAT repetitive elements, and a 282 bp fragment containing 11 GAT repetitive elements;
and/or the primer group of SEQ ID NO: 18 and SEQ ID NO:19 is amplified to obtain a 260 bp fragment containing 7 AT repetitive elements and a 262 bp fragment containing 8 AT repetitive elements.

As a preferred technical solution of the present invention, a reaction system of the fluorescent PCR amplification reaction in step S2 is:
5 μL of 2×Taq PCR Master Mix, 1 μL of genomic DNA, 0.1 μL of forward primer, 0.4 μL of reverse primer, and 0.4 μL of M13 primer with fluorescence, wherein a volume is fixed to 10 μL with sterile deionized water.

Further preferably, the concentrations of the forward primer, the reverse primer and the M13 primer with fluorescence are all 10 uM.

As a preferred technical solution of the present invention, a fluorescent PCR amplification reaction procedure in step S2 is:
performing pre-denaturation at 95° C. for 3 min, denaturation at 95° C. for 30s, PCR annealing during the decrease from 62° ° C. to 55° C. for 30s, and extension at 72° C. for 30s, with a total of 10 cycles; performing denaturation at 95° C. for 30s, annealing at 52° C. for 30s, and extension at 72° C. for 30s, with a total of 25 cycles; performing final extension at 72° C. for 20 min; and after heat preservation at 4° C. for 6h, using the product for fluorescence capillary electrophoresis detection.

Further another purpose of the present invention is to provide application of the DNA barcode and/or the primer group in preparation of a product for recognizing an original place of Floccularia luteovirens.

Further another purpose of the present invention is to provide a product for recognizing an original place of Floccularia luteovirens, which comprises one or more of the above primer groups.

As a preferred technical solution of the present invention, the product is a kit.

According to the above technical solution, compared with the prior art, the present invention discloses and provides the DNA barcode and the primer group for recognizing the original place, which can utilize wild samples of Floccularia luteovirens and a small amount of tissues or hyphae to carry out character breeding of high-quality strains; identification can be carried out at different growth stages such as mycelium, primordia, fruiting bodies and spores; and the present invention has the short detection period, simple operation, no waste, stable and reliable results and good repeatability, and overcomes the shortcomings of traditional breeding methods that the breeding of Floccularia luteovirens strains is not accurate enough, time-consuming and labor-consuming.

Compared with a traditional breeding method and other existing DNA barcode technologies, the present invention has the advantages of time saving, labor saving, money saving, accuracy and high efficiency, plays a positive role in original place identification and genetic breeding of high-quality Floccularia luteovirens, and also provides an effective method for identification and protection of germplasm resources.

### DESCRIPTION OF DRAWINGS

To more clearly describe the technical solutions in the embodiments of the present invention or in the prior art, the drawings required to be used in the description of the embodiments or the prior art will be simply presented below. Apparently, the drawings in the following description are merely the embodiments of the present invention, and for those ordinary skilled in the art, other drawings can also be obtained according to the provided drawings without contributing creative labor.

FIG. 1A is a result diagram of fluorescent PCR amplification using primer 1 in reference example 1 according to the present invention;

FIG. 1B is a result diagram of fluorescent PCR amplification using primer 1 in reference example 2 according to the present invention;

FIG. 1C is a result diagram of fluorescent PCR amplification using primer 1 in test example according to the present invention;

FIG. 2A is a result diagram of fluorescent PCR amplification using primer 2 in reference example 1 according to the present invention;

FIG. 2B is a result diagram of fluorescent PCR amplification using primer 2 in reference example 2 according to the present invention;

FIG. 2C is a result diagram of fluorescent PCR amplification using primer 2 in test example according to the present invention;

FIG. 3A is a result diagram of fluorescent PCR amplification using primer 3 in reference example 1 according to the present invention;

FIG. 3B is a result diagram of fluorescent PCR amplification using primer 3 in reference example 2 according to the present invention;

FIG. 3C is a result diagram of fluorescent PCR amplification using primer 3 in test example according to the present invention;

FIG. 4A is a result diagram of fluorescent PCR amplification using primer 4 in reference example 1 according to the present invention;

FIG. 4B is a result diagram of fluorescent PCR amplification using primer 4 in reference example 2 according to the present invention; and

FIG. 4C is a result diagram of fluorescent PCR amplification using primer 4 in test example according to the present invention.

### DETAILED DESCRIPTION

The technical solutions in the embodiments of the present invention will be clearly and fully described below in combination with the drawings in the embodiments of the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

Embodiments of the present invention disclose a DNA barcode, a primer group and application for recognizing an original place of Floccularia luteovirens. Reagents used are commercially available; and sources thereof are not specifically limited. Unless otherwise mentioned, test methods used are conventional methods.

### Embodiment 1 Establishment of a DNA Barcode of Floccularia luteovirens

Samples of Floccularia luteovirens are collected from Dangxiong County of Tibet Autonomous Region, Qilian County of Qinghai Province and Shiqu County of Sichuan Province and are treated by genome sequencing; and SSR loci in the genome sequences are analyzed by MISA program.

Primers are designed for PCR amplification of the SSR loci; the primers that can amplify the corresponding fragments are reserved; and invalid primers are discarded.

The samples from the above three original places are amplified by effective primers and detected by capillary electrophoresis. The simple sequence repeat (SSR) locus corresponding to original place distinguishment is established by analysis. Finally, four pairs of primers (see Table 1) are obtained. The four pairs of primers are used to amplify the sample genome; and the fragment polymorphism obtained can assist in identifying an original place of Floccularia luteovirens.

TABLE 1
Specific primers for identification of original place of Floccularia luteovirens

| | | Denaturation | | Denaturation | |
|---|---|---|---|---|---|
| | | temperature | | temperature | SSR |
| No. | Forward primer | (° C.) | Reverse primer | (° C.) | element |
| 1 | TGTAAAACGACGGC | 59.966 | AACTGAGAC | 59.965 | GA |
| | CAGTCTTCCCACAGG | | GGAGCAATG | | |
| | CGCCTATAG, as shown | | CA, as shown in | | |
| | in SEQ ID No: 1 | | SEQ ID No: 2 | | |
| 2 | TGTAAAACGACGGC | 60.037 | TTCCTATTAC | 59.896 | TG |
| | CAGTGGCACGTGGTT | | TCGCGGGCA | | |
| | GAGGATGTA, as | | C, as shown in | | |
| | shown in SEQ ID No: 6 | | SEQ ID No: 7 | | |
| 3 | TGTAAAACGACGGC | 60.033 | CCAACAACG | 60.316 | GAT |
| | CAGTTGGGTGATGA | | CGTCACAACT | | |
| | ATTGGAGGGC, as | | C, as shown in | | |
| | shown in SEQ ID No: 11 | | SEQ ID No: 12 | | |
| 4 | TGTAAAACGACGGC | 60.674 | CCAGGAGAA | 59.826 | AT |
| | CAGTAGCCTGTCCGT | | GTACACGAG | | |
| | GTTATGTGC, as shown | | GC, as shown in | | |
| | in SEQ ID No: 18 | | SEQ ID No: 19 | | |

### Embodiment 2 Identification of Original Place of Floccularia luteovirens

(1) The genome of Floccularia luteovirens samples is extracted by an Ezup column fungus genomic DNA extraction kit (No. B518259) of Sangon Biotech (Shanghai) Co., Ltd., and diluted to 20 ng/uL for fluorescent PCR amplification.

(2) Fluorescent PCR amplification of the SSR DNA barcode is carried out with primers in Table 1. An original place of Floccularia luteovirens can be identified by reading DNA barcode information.

Fluorescent PCR amplification reaction system (10 μL): 5 μL of 2×Taq PCR MasterMix, 1 μL of template (genomic DNA), 0.1 μL of forward primer, 0.4 μL of reverse primer (concentrations of the forward primer and the reverse primer are both 10 uM), and 0.4 μL of M13 primer with fluorescence (concentration of 10 uM), wherein a volume is fixed to 10 μL with sterile deionized water.

Reaction conditions: performing pre-denaturation at 95° C. for 3 min, denaturation at 95° C. for 30s, PCR annealing during the decrease from 62° C. to 55° C. for 30s, and extension at 72° C. for 30s, with a total of 10 cycles; performing denaturation at 95° C. for 30s, annealing at 52° C. for 30s, and extension at 72° C. for 30s, with a total of 25 cycles; performing final extension at 72° C. for 20 min; and after heat preservation at 4° C. for 6 h, using the product for fluorescence capillary electrophoresis detection.

(3) After quantitative dilution of the PCR product, 1 μL of a PCR diluted products is denatured with 9 μL of formamide (including 1% internal standard), and then subjected to capillary fluorescence electrophoresis detection by a DNA sequencer ABI 3730×1. The internal standard, which is a molecular weight internal standard (also known as internal lane standard) LIZ-500 bp, is composed of 16 double-stranded DNA fragments labeled with LIZ fluorescein (orange), with molecular weights of 35, 50, 75, 100, 139, 150, 160, 200, 250, 300, 340, 350, 400, 450, 490 and 500 bp respectively. The fragment size in the amplification result electropherogram is equal to the actual bp number of the amplified fragment plus the M13 fluorescent primer about 18 bp (with error of 0-2 bp). The amplified capillary electrophoresis peak is combined with the sequencing result; and the peak number indicates the number of heterozygous amplified fragments of the gene.

(4) The above methods are used to identify Floccularia luteovirens from Qilian County of Qinghai Province, Shiqu County of Sichuan Province and Dangxiong County of Tibet Autonomous Region.

The sample from Dangxiong County of Tibet Autonomous Region is taken as test example; and the samples from Qilian County of Qinghai Province and Shiqu County of Sichuan Province are taken as reference example 1 and reference example 2 respectively.

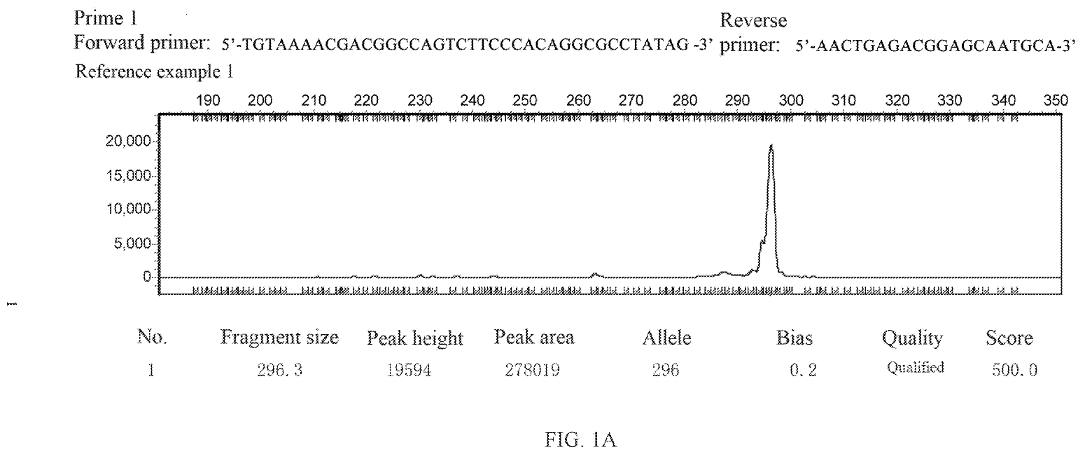

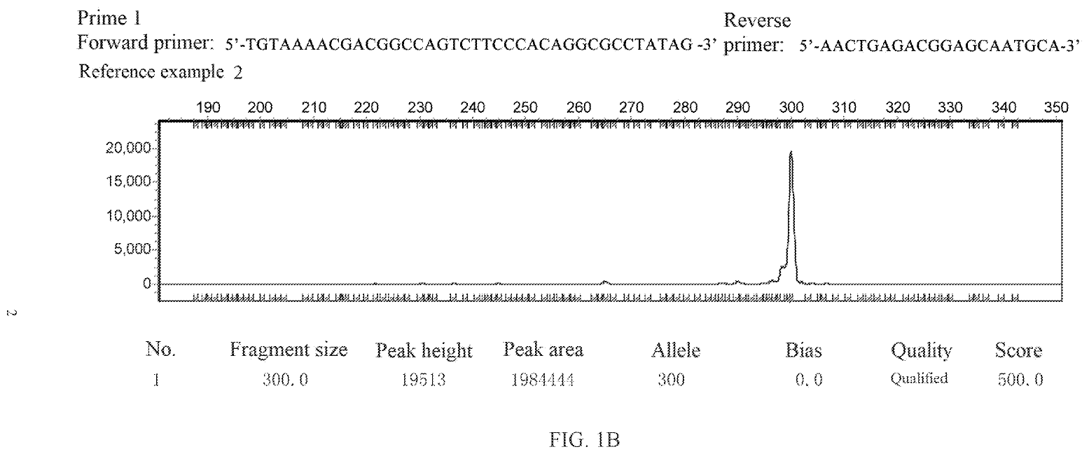

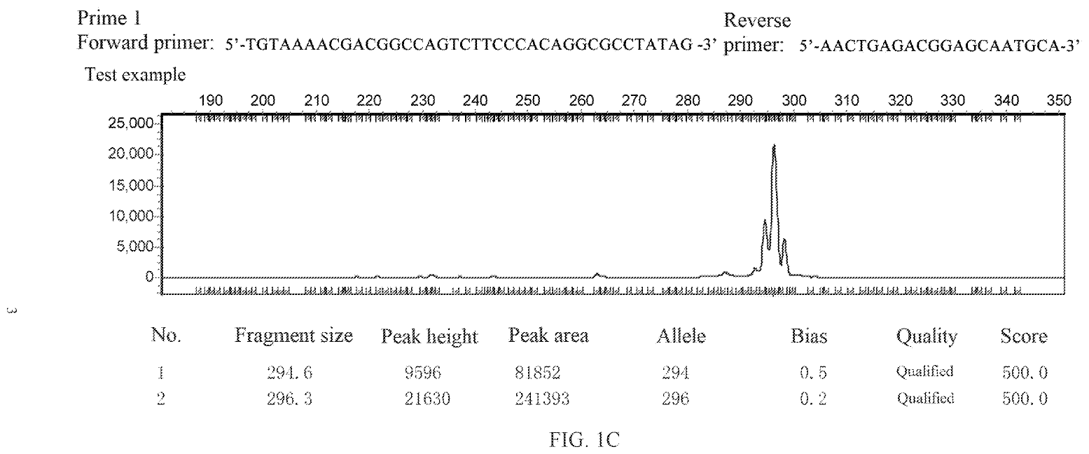

Amplification results of primer 1 are shown in FIGS. 1A-1C. When primer 1 is used for fluorescent PCR amplification, three fragments (three peaks) are obtained through amplification, which contain three SSR loci; and the SSR repetitive element is GA. The amplified fragment obtained in the test example is characterized by containing a 276 bp fragment with 7 repeats and a 278 bp fragment with 8 repeats. In FIGS. 1A-1C, there are non-specific fragments (weak signal small peaks) during amplification of test example. According to sequencing analysis, these fragments do not contain SSR repetitive elements.

Amplified fragments of primer 1: (the statistical fragment length of electropherogram includes the M13 fluorescent primer; the specific sequence shows that the M13 fluorescent primer sequence (18 bp) is removed; and the underlined part is an SSR repetitive element.)

276 bp Amplified Fragment Sequence:

(as shown in SEQ ID NO: 3)
CTTCCCACAGGCGCCTATAGTTGAAGGGCTGAGAGGCAGCTGTGGAGAT
GAAGAAAATTACATACCATCACCAACAATGACGAGCTTCCTTCCTGATC
TATTGAATTAAGAGAGAAAAAGAAGAGAAAGAAAGAGAGAATTGACATA
CAGGGAATTCTGAGAGGCCATTTGAGAGAGAAGATGAGGGAGAGAGAGA
GAGAAAGAAAGAGTTGAGCTATGGAAGCAGACCAAGCAGAACGTCACAG
CCTTCGGTCGCTGCATTGCTCCGTCTCAGTT

278 bp Amplified Fragment Sequence:

(as shown in SEQ ID NO: 4)
CTTCCCACAGGCGCCTATAGTTGAAGGGCTGAGAGGCAGCTGTGGAGAT
GAAGAAAATTACATACCATCACCAACAATGACGAGCTTCCTTCCTGATC
TATTGAATTAAGAGAGAAAAAGAAGAGAAAGAAAGAGAGAATTGACATA
CAGGGAATTCTGAGAGGCCATTTGAGAGAGAAGATGAGGGAGAGAGAGA
GAGAAAGAAAGAGTTGAGCTATGGAAGCAGACCAAGCAGAACGTCACAG
CCTTCGGTCGCTGCATTGCTCCGTCTCAGTT.

282 bp Amplified Fragment Sequence:

(as shown in SEQ ID NO: 5)
CTTCCCACAGGCGCCTATAGTTGAAGGGCTGAGAGGCAGCTGTGGAGAT
GAAGAAAATTACATACCATCACCAACAATGACGAGCTTCCTTCCTGATC
TATTGAATTAAGAGAGAAAAAGAAGAGAAAGAAAGAGAGAATTGACATA
CAGGGAATTCTGAGAGGCCATTTGAGAGAGAAGATGAGGGAGAGAGAGA
GAGAGAGAGAAAGAAAGAGTTGAGCTATGGAAGCAGACCAAGCAGAACG
TCACAGCCTTCGGTCGCTGCATTGCTCCGTCTCAGTT.

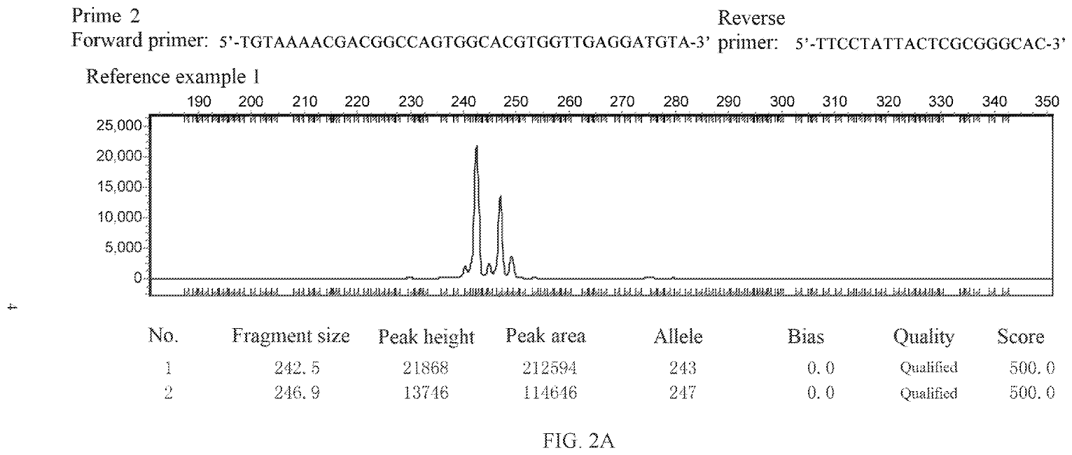

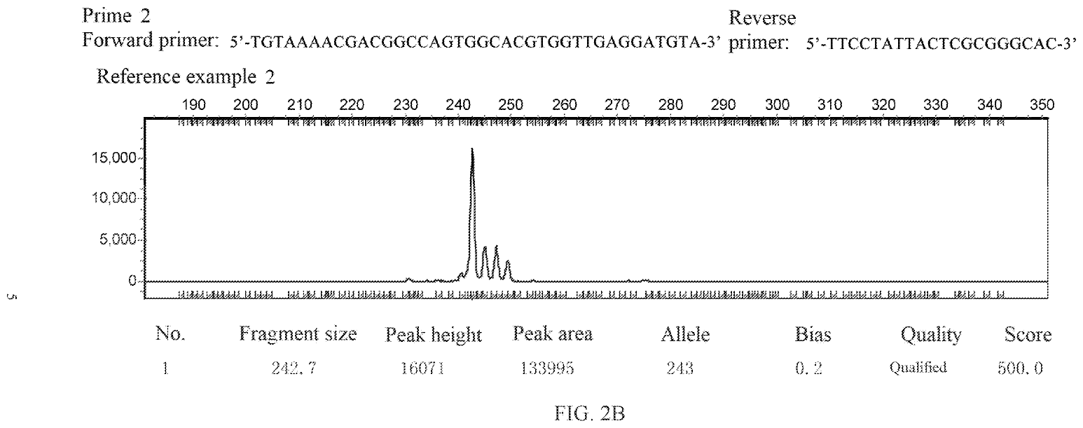

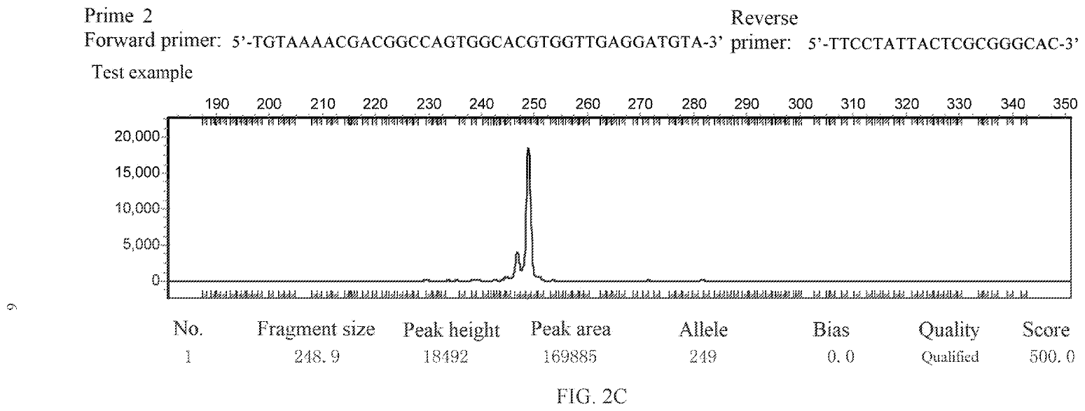

Amplification results of primer 2 are shown in FIGS. 2A-2C. When primer 2 is used for fluorescent PCR amplification, three fragments (three peaks) are obtained, which contain three SSR loci; and the SSR repetitive element is TG. The amplified fragment obtained in the test example is characterized by containing a 231 bp fragment with 12 repeats. In FIGS. 2A-2C, there are non-specific fragments (weak signal small peaks) during amplification of reference example 2. According to sequencing analysis, these fragments do not contain SSR repetitive elements.

Amplified fragments of primer 2: (the statistical fragment length of electropherogram includes the M13 fluorescent primer; the specific sequence shows that the M13 fluorescent primer sequence (18 bp) is removed; and the underlined part is an SSR repetitive element.)

225 bp Amplified Fragment Sequence:

(as shown in SEQ ID NO: 8)
GGCACGTGGTTGAGGATGTATTTGATGTCGACAAGTATTCGGACTTGTG
TGTGTGTGTGTGTGAGAGTGATTTAGAACAGGGATACTTAAGATACATG
AAACTGATGTGGAGACGACAAGAGTTGACGATGAATGGATAAATGAATG
ATGGGACGAGGATGCACAATCCGCGAATGTGGATTGCGCCATGTGACGC
AACGAGGGCGTGCCCGCGAGTAATAGGAA

229 bp Amplified Fragment Sequence:

(as shown in SEQ ID NO: 9)
GGCACGTGGTTGAGGATGTATTTGATGTCGACAAGTATTCGGACTTGTG
TGTGTGTGTGTGTGTGTGAGAGTGATTTAGAACAGGGATACTTAAGATA
CATGAAACTGATGTGGAGACGACAAGAGTTGACGATGAATGGATAAATG
AATGATGGGACGAGGATGCACAATCCGCGAATGTGGATTGCGCCATGTG
ACGCAACGAGGGCGTGCCCGCGAGTAATAGGAA

231 bp Amplified Fragment Sequence:

(as shown in SEQ ID NO: 10)
GGCACGTGGTTGAGGATGTATTTGATGTCGACAAGTATTCGGACTTGTG
TGTGTGTGTGTGTGTGTGTGAGAGTGATTTAGAACAGGGATACTTAAGA
TACATGAAACTGATGTGGAGACGACAAGAGTTGACGATGAATGGATAAA
TGAATGATGGGACGAGGATGCACAATCCGCGAATGTGGATTGCGCCATG
TGACGCAACGAGGGCGTGCCCGCGAGTAATAGGAA

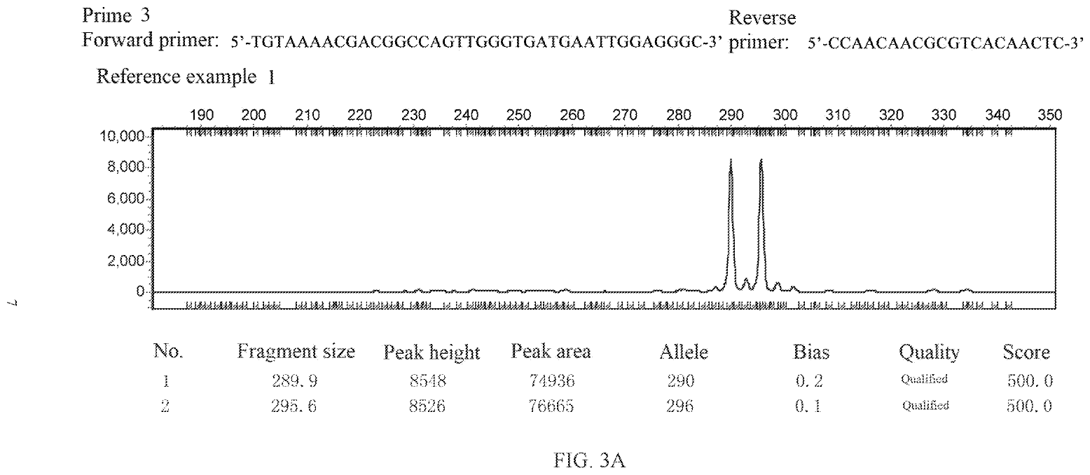

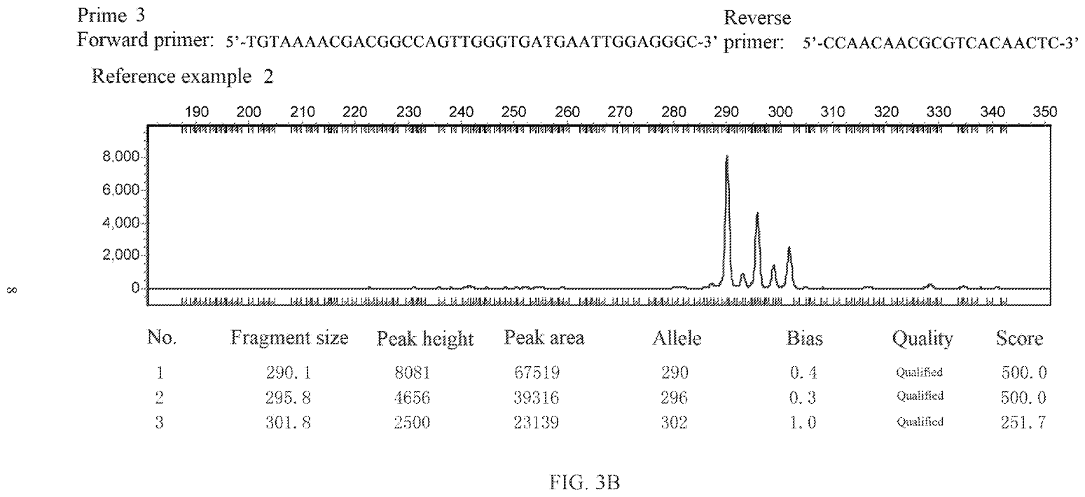

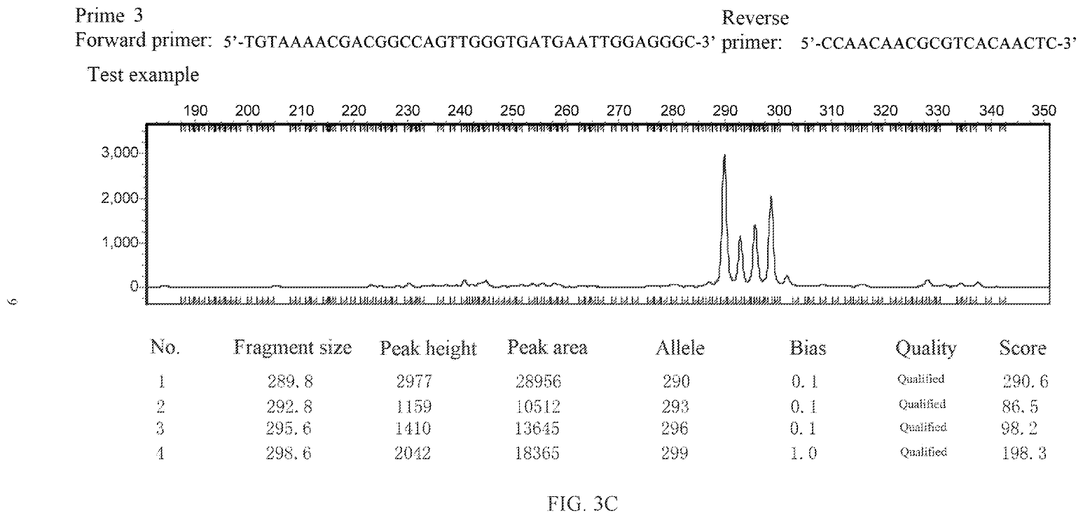

Amplification results of primer 3 are shown in FIGS. 3A-3C. When primer 3 is used for fluorescent PCR amplification, five fragments (five peaks) are obtained, which contain five SSR loci; and the SSR repetitive element is GAT. The amplified fragment obtained in the test example is characterized by containing a 273 bp fragment with 8 repeats, a 276 bp fragment with 9 repeats, a 279 bp fragment with 10 repeats and a 282 bp fragment with 11 repeats.

Amplified fragments of primer 3: (the statistical fragment length of electropherogram includes the M13 fluorescent primer; the specific sequence shows that the M13 fluorescent primer sequence (17 bp) is removed with an error of 1 bp; and the underlined part is an SSR repetitive element.)

273 bp Amplified Fragment Sequence:

(as shown inSEQ ID NO: 13)
TGGGTGATGAATTGGAGGGCTCGTATGTGGGCGTTTATTAAGATGTCAT
CTGATGAGGTAGGGTCGAGACACCATTCTTCGAAAGAAGGGAGACCGGG
TGCATCCCATATTTGGGAAAGTGAGTATCTAGCTAGGTCTTTTGCAGCC
ATTGATGTTGAACTTGAGCCTGCAGGAGATGATGATGATGATGATGATG
ATGGTGTGGTTATTATACAGTGTGGGCATTGACAATGGAAACGGTATTG
GTCGTATAGAGTTGTGACGCGTTGTTGG

276 bp Amplified Fragment Sequence:

(as shown in SEQ ID NO: 14)
TGGGTGATGAATTGGAGGGCTCGTATGTGGGCGTTTATTAAGATGTCAT
CTGATGAGGTAGGGTCGAGACACCATTCTTCGAAAGAAGGGAGACCGGG
TGCATCCCATATTTGGGAAAGTGAGTATCTAGCTAGGTCTTTTGCAGCC
ATTGATGTTGAACTTGAGCCTGCAGGAGATGATGATGATGATGATGATG
ATGATGGTGTGGTTATTATACAGTGTGGGCATTGACAATGGAAACGGTA
TTGGTCGTATAGAGTTGTGACGCGTTGTTGG

279 bp Amplified Fragment Sequence:

(as shown in SEQ ID NO: 15)
TGGGTGATGAATTGGAGGGCTCGTATGTGGGCGTTTATTAAGATGTCAT
CTGATGAGGTAGGGTCGAGACACCATTCTTCGAAAGAAGGGAGACCGGG
TGCATCCCATATTTGGGAAAGTGAGTATCTAGCTAGGTCTTTTGCAGCC
ATTGATGTTGAACTTGAGCCTGCAGGAGATGATGATGATGATGATGATG
ATGATGATGGTGTGGTTATTATACAGTGTGGGCATTGACAATGGAAACG
GTATTGGTCGTATAGAGTTGTGACGCGTTGTTGG

282 bp Amplified Fragment Sequence:

(as shown in SEQ ID NO: 16)
TGGGTGATGAATTGGAGGGCTCGTATGTGGGCGTTTATTAAGATGTCAT
CTGATGAGGTAGGGTCGAGACACCATTCTTCGAAAGAAGGGAGACCGGG
TGCATCCCATATTTGGGAAAGTGAGTATCTAGCTAGGTCTTTTGCAGCC
ATTGATGTTGAACTTGAGCCTGCAGGAGATGATGATGATGATGATGATG
ATGATGATGATGGTGTGGTTATTATACAGTGTGGGCATTGACAATGGAA
ACGGTATTGGTCGTATAGAGTTGTGACGCGTTGTTGG

285 bp Amplified Fragment Sequence:

(as shown in SEQ ID NO: 17)
TGGGTGATGAATTGGAGGGCTCGTATGTGGGCGTTTATTAAGATGTCAT
CTGATGAGGTAGGGTCGAGACACCATTCTTCGAAAGAAGGGAGACCGGG
TGCATCCCATATTTGGGAAAGTGAGTATCTAGCTAGGTCTTTTGCAGCC
ATTGATGTTGAACTTGAGCCTGCAGGAGATGATGATGATGATGATGATG
ATGATGATGATGATGGTGTGGTTATTATACAGTGTGGGCATTGACAATG
GAAACGGTATTGGTCGTATAGAGTTGTGACGCGTTGTTG

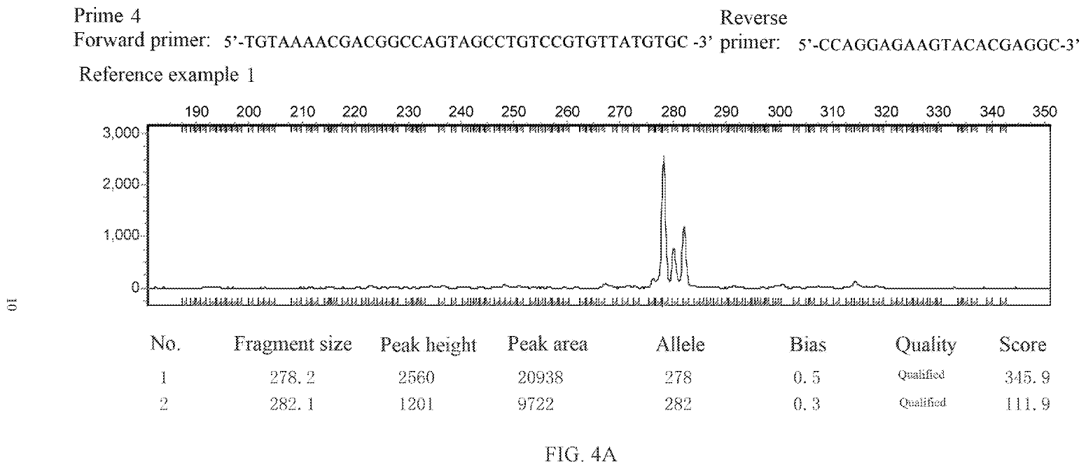

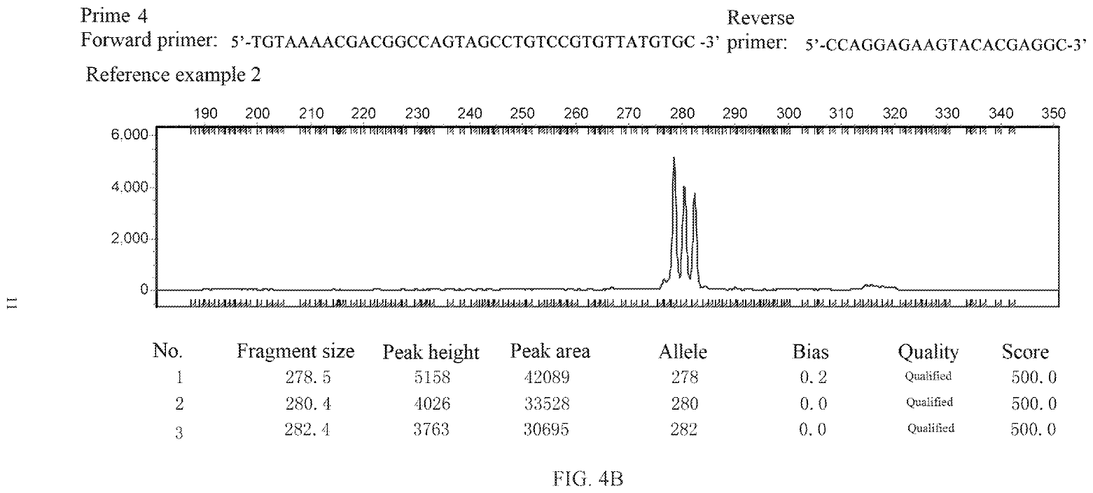

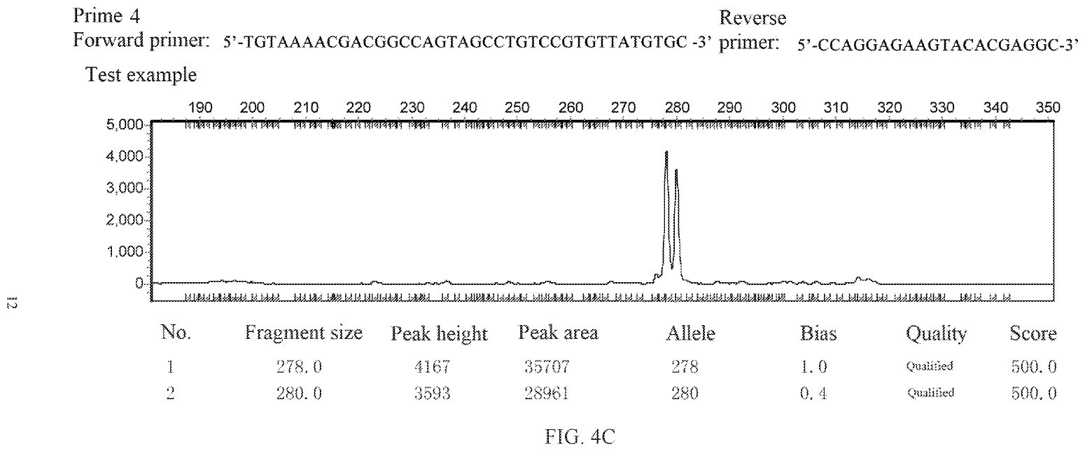

Amplification results of primer 4 are shown in FIGS. 4A-4C. When primer 4 is used for fluorescent PCR amplification, three fragments (three peaks) are obtained, which contain three SSR loci; and the SSR repetitive element is AT. The amplified fragment obtained in the test example is characterized by containing a 260 bp fragment with 7 repeat and a 262 bp fragment with 8 repeats.

Amplified fragments of primer 4: (the statistical fragment length of electropherogram includes the M13 fluorescent primer; the specific sequence shows that the M13 fluorescent primer sequence (18 bp) is removed with an error of 1 bp; and the underlined part is an SSR repetitive element.)

260 bp Amplified Fragment Sequence:

(as shown in SEQ ID NO: 20)
AGCCTGTCCGTGTTATGTGCAAACGGAAACATAGTAAAAATAGGTATTA
CTGTACATGGTATTAATTTATTTTGTGAAATGTTAGTGCAATGATATAT
ATATATATAAGTACTGTAGATCATCTACTTATAAAAGGTGATATTTGAC
ATTAGCAGTACACGTATAGCGAATACCAGTTTGCTGTCATGAGAAAGAC
GAGAAGATATCAAGCTACTTTAATGATACCGAATACGTTGAAACGCCTC
GTGTACTTCTCCTGG

262 bp Amplified Fragment Sequence:

(as shown in SEQ ID NO: 21)
AGCCTGTCCGTGTTATGTGCAAACGGAAACATAGTAAAAATAGGTATTA
CTGTACATGGTATTAATTTATTTTGTGAAATGTTAGTGCAATGATATAT
ATATATATATAAGTACTGTAGATCATCTACTTATAAAAGGTGATATTTG
ACATTAGCAGTACACGTATAGCGAATACCAGTTTGCTGTCATGAGAAAG
ACGAGAAGATATCAAGCTACTTTAATGATACCGAATACGTTGAAACGCC
TCGTGTACTTCTCCTGG

264 bp Amplified Fragment Sequence:

(as shown in SEQ ID NO: 22)
AGCCTGTCCGTGTTATGTGCAAACGGAAACATAGTAAAAATAGGTATTA
CTGTACATGGTATTAATTTATTTTGTGAAATGTTAGTGCAATGATATAT
ATATATATATATAAGTACTGTAGATCATCTACTTATAAAAGGTGATATT
TGACATTAGCAGTACACGTATAGCGAATACCAGTTTGCTGTCATGAGAA
AGACGAGAAGATATCAAGCTACTTTAATGATACCGAATACGTTGAAACG
CCTCGTGTACTTCTCCTGG

Through comprehensive analysis of maps and sequencing results, the characteristic information of the DNA barcode of Floccularia luteovirens with different original places is shown in Table 2:

Floccularia luteovirens with Qilian County of Qinghai Province as the original place is characterized in that: primer 1 carries out amplification to obtain the 278 bp fragment containing 8 GA repetitive elements (as shown in SEQ ID NO:3); primer 2 carries out amplification to obtain the 225 bp fragment containing 9 TG repetitive elements (as shown in SEQ ID NO:8) and the 229 bp fragment containing 11 TG repetitive elements (as shown in SEQ ID NO:9); primer 3 carries out amplification to obtain the 273 bp fragment containing 8 GAT repetitive elements (as shown in SEQ ID NO:13) and the 279 bp fragment containing 10 GAT repetitive elements (as shown in SEQ ID NO:14); and primer 4 carries out amplification to obtain the 260 bp fragment containing 7 AT repetitive elements (as shown in SEQ ID NO:20) and the 264 bp fragment containing 9 AT repetitive elements (as shown in SEQ ID NO:21).

Floccularia luteovirens with Shiqu County of Sichuan Province as the original place is characterized in that: primer 1 carries out amplification to obtain the 282 bp fragment containing 10 GA repetitive elements (as shown in SEQ ID NO:4); primer 2 carries out amplification to obtain the 225 bp fragment containing 9 TG repetitive elements (as shown in SEQ ID NO:8); primer 3 carries out amplification to obtain the 273 bp fragment containing 8 GAT repetitive elements (as shown in SEQ ID NO:13), the 279 bp fragment containing 10 GAT repetitive elements (as shown in SEQ ID NO:14) and the 285 bp fragment containing 12 GAT repetitive elements (as shown in SEQ ID NO:15); and primer 4 carries out amplification to obtain the 260 bp fragment containing 7 AT repetitive elements (as shown in SEQ ID NO:20), the 262 bp fragment containing 8 AT repetitive elements (as shown in SEQ ID NO:22) and the 264 bp fragment containing 9 AT repetitive elements (as shown in SEQ ID NO:21).

Floccularia luteovirens with Dangxiong County of Tibet Autonomous Region as the original place is characterized in that: primer 1 carries out amplification to obtain the 276 bp fragment containing 7 GA repetitive elements (as shown in SEQ ID NO:5) and the 278 bp fragment containing 8 GA repetitive elements (as shown in SEQ ID NO:3); primer 2 carries out amplification to obtain the 231 bp fragment containing 12 TG repetitive elements (as shown in SEQ ID NO:10); primer 3 carries out amplification to obtain the 273 bp fragment containing 8 GAT repetitive elements (as shown in SEQ ID NO:13), the 276 bp fragment containing 9 GAT repetitive elements (as shown in SEQ ID NO:16), the 279 bp fragment containing 10 GAT repetitive elements (as shown in SEQ ID NO:14) and the 282 bp fragment containing 11 GAT repetitive elements (as shown in SEQ ID NO:17); and primer 4 carries out amplification to obtain the 260 bp fragment containing 7 AT repetitive elements (as shown in SEQ ID NO:20), and the 262 bp fragment containing 8 AT repetitive elements (as shown in SEQ ID NO:22).

TABLE 2
DNA barcode characteristic information of Floccularia luteovirens with
different original places

| | Primer 1 | Primer 2 | Primer 3 | Primer 4 |
|---|---|---|---|---|
| | amplified | amplified | amplified | amplified |
| | fragment | fragment | fragment | fragment |
| Original place | (SSR) | (SSR) | (SSR) | (SSR) |
| Qilian County of | 278 bp (8GA) | 225 bp (9TG) | 273 bp (8GAT) | 260 bp (7AT) |
| Qinghai Province | | 229 bp (11TG) | 279 bp (10GAT) | 264 bp (9AT) |
| Shiqu County of | 282 bp (10GA) | 225 bp (9TG) | 273 bp (8GAT) | 260 bp (7AT) |
| Sichuan Province | | | 279 bp (10GAT) | 262 bp (8AT) |
| | | | 285 bp (12GAT) | 264 bp (9AT) |
| Dangxiong County | 276 bp (7GA) | 231 bp (12TG) | 273 bp (8GAT) | 260 bp (7AT) |
| of Tibet | 278 bp (8GA) | | 276 bp (9GAT) | 262 bp (8AT) |
| Autonomous Region | | | 279 bp (10GAT) | |
| | | | 282 bp (11GAT) | |

### Embodiment 3 Verification of DNA Barcode for Identifying Original Place of Floccularia luteovirens

A DNA barcode for identifying an original place of Floccularia luteovirens is verified by blind testing.

Step 1, blind testing: taking 16 samples from Qilian County of Qinghai Province (original place No. 1-16), 16 samples from Shiqu County of Sichuan Province (original place No. 17-32) and 16 samples from Dangxiong County of Tibet Autonomous Region (original place No. 33-48) for blind testing;

step 2, testing: using primers (SEQ ID NO:1, SEQ ID NO:2, SEQ ID NO:6, SEQ ID NO:7, SEQ ID NO:11, SEQ ID NO: 12, SEQ ID NO: 18 and SEQ ID NO: 19) for amplification and capillary electrophoresis. One or more pairs of primer groups can be combined for amplification; and DNA barcode characteristics can be used to distinguish the blind testing samples and identify the samples with the different original places;

step 3, unblinding: results are shown in Table 3. The unblinding identification results of 16 samples from Qilian County of Qinghai Province, Shiqu County of Sichuan Province and Dangxiong County of Tibet Autonomous Region are all correct according to the DNA barcode characteristics, indicating that the DNA barcode is suitable for identifying an original place.

TABLE 3
Unblinding original place identification results based on DNA barcode characteristics of original places

| | Primer 1 | Primer 2 | Primer 3 | Primer 4 | Unblinding | |
|---|---|---|---|---|---|---|
| Blind | amplified | amplified | amplified | amplified | original | Identification |
| testing No. | fragment (SSR) | fragment (SSR) | fragment (SSR) | fragment (SSR) | place No. | results |
| 3, 10, 11, 18, | 278 bp (8GA) | 225 bp (9TG) | 273 bp (8GAT) | 260 bp (7AT) | 1-16 | Qilian County of |
| 21, 23, 27, 29, | | 229 bp (11TG) | 279 bp (10GAT) | 264 bp (9AT) | | Qinghai Province |
| 31, 32, 36, 37, | | | | | | |
| 40, 41, 44, 48 | | | | | | |
| 1, 6, 9, 13, 16, | 282 bp (10GA) | 225 bp (9TG) | 273 bp (8GAT) | 260 bp (7AT) | 17-32 | Shiqu County of |
| 17, 20, 24, 28, | | | 279 bp (10GAT) | 262 bp (8AT) | | Sichuan Province |
| 30, 33, 38, 39, | | | 285 bp (12GAT) | 264 bp (9AT) | | |
| 42, 46, 47 | | | | | | |
| 2, 4, 5, 7, 8, 12, | 276 bp (7GA) | 231 bp (12TG) | 273 bp (8GAT) | 260 bp (7AT) | 33-48 | Dangxiong |
| 14, 15, 19, 22, 25, | 278 bp (8GA) | | 276 bp (9GAT) | 262 bp (8AT) | | County of Tibet |
| 26, 34, 35, 43, 45, | | | 279 bp (10GAT) | | | Autonomous Region |

Each embodiment in the description is described in a progressive way. The difference of each embodiment from each other is the focus of explanation. The same and similar parts among all of the embodiments can be referred to each other.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. A DNA barcode for recognizing an original place of Floccularia luteovirens, wherein a nucleotide sequence of the DNA barcode comprises one or more of:
SEQ ID NO:4,
and/or SEQ ID NO:5,
and/or a combination of SEQ ID NO:3 and SEQ ID NO:4,
and/or a combination of SEQ ID NO:8 and SEQ ID NO:9,
and/or SEQ ID NO:8,
and/or SEQ ID NO:10,
and/or a combination of SEQ ID NO:13 and SEQ ID NO:15,
and/or a combination of SEQ ID NO:13, SEQ ID NO:15 and SEQ ID NO:17,
and/or a combination of SEQ ID NO:13, SEQ ID NO:14, SEQ ID NO:15 and SEQ ID NO:16,
and/or a combination of SEQ ID NO:20 and SEQ ID NO:22,
and/or a combination of SEQ ID NO:20, SEQ ID NO:21 and SEQ ID NO:22,
and/or a combination of SEQ ID NO:20 and SEQ ID NO:21.

2. A primer group for amplifying the DNA barcode for recognizing the original place of Floccularia luteovirens according to claim 1, wherein a nucleotide sequence of the primer group comprises one or more groups of:
SEQ ID NO: 1 and SEQ ID NO:2,
and/or SEQ ID NO:6 and SEQ ID NO:7,
and/or SEQ ID NO:11 and SEQ ID NO:12,
and/or SEQ ID NO:18 and SEQ ID NO:19.

3. The primer group according to claim 2, wherein the nucleotide sequence of the primer group comprises:
such as SEQ ID NO: 1 and SEQ ID NO:2,
and SEQ ID NO:6 and SEQ ID NO:7,
and SEQ ID NO:11 and SEQ ID NO:12,
and SEQ ID NO:18 and SEQ ID NO:19.

4. A method for recognizing an original place of Floccularia luteovirens, comprising the following steps:
S1, extracting genomic DNA of a sample to be tested;
S2, taking the genomic DNA in S1 as a template, and performing a fluorescent PCR amplification reaction on one or more groups of the primers according to claim 2 to obtain an amplification product;
S3, detecting the amplification product of S2 by capillary fluorescence electrophoresis, and judging by the number of fragments, the number of SSR loci, SSR repetitive elements and repeating times of the amplification product.

5. The method for recognizing the original place of Floccularia luteovirens according to claim 4, wherein a judgment standard in step S3 is:
(1) a DNA barcode of Floccularia luteovirens with Qilian County of Qinghai Province as the original place is characterized in that: the primer group of SEQ ID NO:1 and SEQ ID NO:2 is amplified to obtain a 278 bp fragment containing 8 GA repetitive elements;
and/or the primer group of SEQ ID NO:6 and SEQ ID NO:7 is amplified to obtain a 225 bp fragment containing 9 TG repetitive elements and a 229 bp fragment containing 11 TG repetitive elements;
and/or the primer group of SEQ ID NO:11 and SEQ ID NO:12 is amplified to obtain a 273 bp fragment containing 8 GAT repetitive elements and a 279 bp fragment containing 10 GAT repetitive elements;
and/or the primer group of SEQ ID NO:18 and SEQ ID NO:19 is amplified to obtain a 260 bp fragment containing 7 AT repetitive elements and a 264 bp fragment containing 9 AT repetitive elements;
(2) a DNA barcode of Floccularia luteovirens with Shiqu County of Sichuan Province as the original place is characterized in that: the primer group of SEQ ID NO: 1 and SEQ ID NO:2 is amplified to obtain a 282 bp fragment containing 10 GA repetitive elements;
and/or the primer group of SEQ ID NO:6 and SEQ ID NO:7 is amplified to obtain a 225 bp fragment containing 9 TG repetitive elements;
and/or the primer group of SEQ ID NO:11 and SEQ ID NO:12 is amplified to obtain a 273 bp fragment containing 8 GAT repetitive elements, a 279 bp fragment containing 10 GAT repetitive elements and a 285 bp fragment containing 12 GAT repetitive elements;
and/or the primer group of SEQ ID NO:18 and SEQ ID NO:19 is amplified to obtain a 260 bp fragment containing 7 AT repetitive elements, a 262 bp fragment containing 8 AT repetitive elements and a 264 bp fragment containing 9 AT repetitive elements;
(3) a DNA barcode of Floccularia luteovirens with Dangxiong County of Tibet Autonomous Region as the original place is characterized in that: the primer group of SEQ ID NO:1 and SEQ ID NO:2 is amplified to obtain a 276 bp fragment containing 7 GA repetitive elements, and a 278 bp fragment containing 8 GA repetitive elements;
and/or the primer group of SEQ ID NO:6 and SEQ ID NO:7 is amplified to obtain a 231 bp fragment containing 12 TG repetitive elements;
and/or the primer group of SEQ ID NO:11 and SEQ ID NO:12 is amplified to obtain a 273 bp fragment containing 8 GAT repetitive elements, a 276 bp fragment containing 9 GAT repetitive elements, a 279 bp fragment containing 10 GAT repetitive elements, and a 282 bp fragment containing 11 GAT repetitive elements;
and/or the primer group of SEQ ID NO:18 and SEQ ID NO:19 is amplified to obtain a 260 bp fragment containing 7 AT repetitive elements and a 262 bp fragment containing 8 AT repetitive elements.

6. The method for recognizing the original place of Floccularia luteovirens according to claim 4, wherein a reaction system of the fluorescent PCR amplification reaction in step S2 is:
5 μL of 2×Taq PCR Master Mix, 1 μL of genomic DNA, 0.1 μL of forward primer, 0.4 μL of reverse primer, and 0.4 μL of M13 primer with fluorescence, wherein a volume is fixed to 10 μL with sterile deionized water.

7. The method for recognizing the original place of Floccularia luteovirens according to claim 6, wherein the concentrations of the forward primer, the reverse primer and the M13 primer with fluorescence are all 10 uM.

8. The method for recognizing the original place of Floccularia luteovirens according to claim 4, wherein a fluorescent PCR amplification reaction procedure in step S2 is:
performing pre-denaturation at 95° C. for 3 min, denaturation at 95° ° C. for 30s, PCR annealing during the decrease from 62° C. to 55° C. for 30s, and extension at 72° C. for 30s, with a total of 10 cycles; performing denaturation at 95° C. for 30s, annealing at 52° C. for 30s, and extension at 72° C. for 30s, with a total of 25 cycles; performing final extension at 72° C. for 20 min; and after heat preservation at 4° C. for 6 h, using the product for fluorescence capillary electrophoresis detection.

9. An application of the DNA barcode according to claim 1 in preparation of a product for recognizing an original place of Floccularia luteovirens.

10. An application of the primer group according to claim 2 in preparation of a product for recognizing an original place of Floccularia luteovirens.

11. A product for recognizing an original place of Floccularia luteovirens, comprising one or more of the primer groups according to claim 2.
